(19) United States
(12) Patent Application Publication
(10) Pub. No.: US 2007/0016273 A1
(43) Pub. Date: Jan. 18, 2007

(54) ELECTROTHERMAL INTERVERTEBRAL DISC TREATMENT

(75) Inventors: Nelson Scarborough, Andover, MA (US); Kamran Behzadian, Sunnyvale, CA (US); Ralph I. McNall, Belmont, CA (US)

Correspondence Address: FISH & RICHARDSON P.C.;SMITH & NEPHEW, INC., 1450 BROOKS ROAD, MEMPHIS, TN, 38116, US

(73) Assignee: SMITH & NEPHEW, INC., Memphis, TN (US)

(21) Appl. No.: 11/420,673

(22) Filed: May 26, 2006

Related U.S. Application Data
(60) Provisional application No. 60/684,518, filed on May 26, 2005.

(51) Int. Cl. A61F 7/00; A61F 7/12

(52) U.S. Cl. 607/96; 607/99; 607/113

(57) ABSTRACT

A method of treating an intervertebral disc includes actively steering a heat-delivery device in two dimensions to a region of a treatment site within the intervertebral disc, and heating the region of the treatment site. A heating element of the heat-delivery device includes various configurations to provide a large heating profile in order to heat, and cause denervation at, substantially all of the region of the treatment site.

### TECHNICAL FIELD

This invention relates to electrothermal intervertebral disc treatment.

### BACKGROUND

Discogenic back pain is believed to be caused by disc degeneration characterized by fissures within an intervertebral disc. Evidence that the disc itself can be a source of pain has been provided by studies that have performed disc probing to elicit a pain response from a patient in an aware state. A correlation is also seen between disc pain and anatomical changes consistent with fissures in the disc.

The primary diagnostic tool used in these studies is discography. In discography, a needle is inserted into a disc to inject saline and contrast media into the disc. By pressurizing the disc, a pain response is elicited, analogous to palpation. The contrast media can be visualized using fluoroscopy to evaluate the anatomy of the disc. Discs are graded on a scale of 1 to 4 to indicate the extent of degeneration, where the higher the number, the greater the extent of degeneration.

When a disc undergoes the natural process of degeneration, changes that occur in the architecture and biochemical environment of the disc may lead to altered loading patterns of weight upon the disc, and also may lead to sensitization of nociceptor nerve fibers. The combination of mechanical and neuronal changes leads to chronic discogenic pain.

Various researchers have demonstrated the existence of nociceptors within a disc. When a disc develops a fissure, a repair process ensues that is characterized by blood vessels growing into the outer annulus of the disc. Along with this vascularization, innervation occurs. This innervation results in loading of pain receptors within the annulus such that under normal loading conditions for a disc, the pain receptors cause discogenic back pain.

Discogenic back pain results in pain around the effected vertebrae, as well as referral of pain to a broader area, such as the buttocks and thighs. This type of pain is distinguishable from radicular pain, which is typically a shooting pain that radiates down the leg to the calf. Radicular pain may occur due to impingement of nerve roots as the nerve roots exit the spine. Impingement of nerve roots often occurs due to bulging or herniation of a disc resulting in compression and sensitization of the nerve root as it exits the foramen. Discogenic and radicular pain may occur in the cervical spine as well as the lumbar spine. Discogenic and radicular symptoms are often coexistent and not clearly distinguishable in many patients.

Various current treatments for back pain range from conservative management (e.g., exercise and/or anti-inflammatory drug therapy) to surgical procedures such as spine fusion or arthrodesis, and arthroplasty. In the surgical procedures, the goal is typically to remove an offending disc and either fuse the segment where the disc had been located or replace the disc with an artificial disc.

Non-surgical alternatives, such as Intradiscal Electrothermal Therapy (IDET), have been developed, at least in part, as a way to relieve back pain by heating the painful disc. The pain may be relieved, for example, by denervating (that is, killing) the pathological nerve tissue in the disc, without having to resort to surgery. More particularly, heat applied to the region of a fissure modifies the collagen structure around the fissure to create a lesion. Heating of the tissue to temperatures greater than 45° C. is typically required to kill nerve tissue and create the lesion.

### SUMMARY

In a general aspect, a method for treating an intervertebral disc includes actively steering a heat-delivery device to a region of a treatment site within the intervertebral disc. The treatment site is three dimensional and the steering navigates the heat-delivery device in at least two dimensions. The method includes applying heat to the region of the treatment site using the heat-delivery device.

Implementations of this aspect may include one or more of the following features. For example, actively steering the heat-delivery device includes turning a knob positioned external to a patient to navigate the heat-delivery device in two dimensions. Alternatively, actively steering the heat-delivery device includes activating a handle positioned external to a patient to navigate the heat-delivery device in two dimensions.

The heat-delivery device includes a guide wire and a heating element and the guide wire is actively steered to the region of the treatment site within the intervertebral disc and the heating element is advanced to the region of the treatment site over the guide wire.

Alternatively, the heat-delivery device includes a sheath and a heating element and the sheath is actively steered to the region of the treatment site within the intervertebral disc and the heating element is advanced to the region of the treatment site through the sheath. The heating element can be removed from the sheath and a second device, having a function other than heat-delivery, can be advanced to the treatment site through the sheath.

Applying heat to the region of the treatment site includes applying heat to substantially all of the treatment site using, for example, a heating element of the heat-delivery device having a three dimensional shape that corresponds to a three dimensional shape of the treatment site. Heat can be applied to the region of the treatment site at a location that is at a distance from the treatment site. A conductive material, for example, saline, can be injected into the treatment site and heat applied to the conductive material in the treatment site.

The temperature of the heat applied to the treatment site can be monitored by placing a thermocouple at the outer wall of the annulus of the intervertebral disc to monitor the temperature of the heat applied, for example, to the inner wall of the annulus of the intervertebral disc.

Actively steering a heat-delivery device to the region of the treatment site includes actively steering the heat-delivery device to a location at a distance from the treatment site and placing a thermocouple at a distance from the heat-delivery device that mimics the distance from the heat-delivery device to the treatment site to monitor the temperature of the heat applied to the treatment site.

Monitoring the temperature includes navigating a thermocouple to a location for monitoring temperature separately from the navigating of the heat-delivery device.

Applying heat to the treatment site includes applying heat at a temperature greater than 45° C.

Applying heat to the treatment site includes applying heat to the treatment site with a heating element of the heat-delivery device in a monopolar or bipolar configuration. The heating element includes, for example, electrodes formed into a basket configuration, or an electrode that is coiled inside a sheath in an undeployed state and is extended into the intervertebral disc to form a flat shape in a deployed shape. Alternatively, the heating element includes at least two electrodes and a distance between the electrodes is constant along the length of the electrodes.

A fluid can be injected into the treatment site, and the heating element can be configured to inject the fluid into the treatment site.

In another general aspect, a device for intervertebral disc treatment includes a heat-delivery device configured to encompass a three dimensional volume of the disc to provide heat to a three dimensional treatment site within the intervertebral disc. The device includes an active steering mechanism configured to enable navigation of the heat-delivery device in at least two dimensions.

Implementations of this aspect may include one or more of the following features. For example, the steering mechanism includes a pre-bent guide wire and a knob for rotating the guide wire about its longitudinal axis. Alternatively or additionally, the steering mechanism includes a guide catheter and pull wires or strips for navigating the guide catheter. Various means for controlling the pull wires or strips are disclosed.

The guide catheter can be navigated by selectively applying electricity to a conductive fluid contained within the guide catheter.

The heat-delivery device includes a heating member that expands when deployed within the disc to encompass the three dimensional volume of the disc.

The heating member includes electrodes that fan out in the deployed state, are in the shape of a basket, or uncoil when deployed.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A illustrates the three-dimensional nature of disc fissures.

FIG. 1B is a cross-sectional view of a disc with a complex radial and concentric tear being treated by a heat-delivery device.

FIGS. 2A, 2B and 2C illustrate an active steering mechanism for navigating a guide wire of a heat-delivery device to a region of a disc.

FIGS. 3-8 illustrate active steering mechanisms for navigating a guide catheter of a heat-delivery device to a region of a disc.

FIGS. 9A and 9B illustrate a heating member of a heat-delivery device.

FIGS. 10-16 illustrate other embodiments of a heating member of a heat-delivery device.

FIG. 17 illustrates the introduction of electrically conductive materials into a treatment site.

FIG. 18 illustrates a device that introduces electrically conductive materials into a treatment site.

Like reference symbols in the various drawings indicate like elements.

### DETAILED DESCRIPTION

Techniques are described for treating pathology in an intervertebral disc. The pathology, such as a fissure in the annular wall of the intervertebral disc, is treated by actively steering a heat-delivery device to a treatment site within the intervertebral disc. The heat-delivery device can include a heating element that provides a heating profile that substantially covers the pathology. A combination of these two features provides targeted placement of a device at a site of a pathology and a heating profile that treats substantially the entire pathology. Such combinations are desirable due to the three-dimensional structure of an intervertebral disc and the localized nature of a disc pathology. The treatment site can be in a region of a fissure (in patients with discogenic pain) or a herniation (in patients with radicular symptoms). Other functions that can be performed within the disc in targeted locations include the addition or removal of material, and visualization and penetration of dense tissue to access a treatment site.

Fluoroscopy can be used to aid in navigating the device to the treatment site. Because the treatment site is typically three-dimensional, active steering of the device is provided in at least two dimensions, for example, in the up and down directions and the side-to-side directions relative to the device axis. The active steering, combined with non-steering advancement and retraction of the device along the device axis, provides the navigability to precisely position the distal end of the device as desired within the disc.

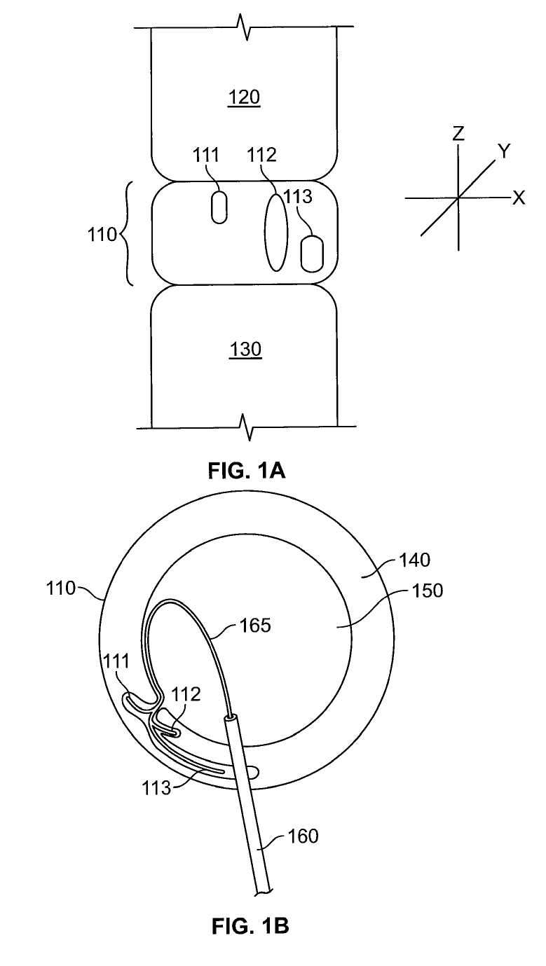

Referring to FIGS. 1A and 1B, three fissures 111-113 are shown within an intervertebral disc 110 located between two vertebral bodies 120 and 130. To treat the fissures, a heat-delivery device 165 that can be actively steered to reach any location within disc 110 is inserted into disc 110 and activated to apply heat to treat the fissures. In one example, a heat-delivery device is actively steered to a location within disc 110 to treat a portion of, for example, fissure 112. The heat-delivery device is then repositioned (also using active steering) to treat another portion of fissure 112. This process is repeated until all three fissure are treated. In another example, a heat-delivery device is actively steered to a location within disc 110 to provide heat to treat an entire fissure, such as, for example, fissure 112, at once, or multiple fissures, such as fissure 111 and 112 and/or 113, at once.

In FIG. 1B, the three fissures 111-113 are shown extending from nucleus pulposus 150 into the annular wall 140 in intervertebral disc 110. As illustrated, fissure 111 is a radial tear and fissures 112 and 113 are concentric tears.

The heat delivery device 165 is introduced into the disc through an introducer needle 160 and actively steered such that the distal portion of the device 165 is navigated near, or into, any one or more of fissures 111-113. For example, device 165 can be actively steered such that the distal portion of the device follows the full length of a concentric tear.

FIGS. 1A and 1B demonstrate the three dimensional nature of disc fissures. The ability to actively steer a device up and down (along the Z-axis in FIG. 1A) and side-to-side (along the Y-axis in FIG. 1A), as well as advance and retract the device along the device axis (along the X-axis in FIG. 1A) provides the desired degrees of freedom to place the device relative to the fissures. The device need not be separately steerable along the directions of the Y and Z axes, as long as the device can be navigated in two-dimensions in a plane defined by the Y and Z axes.

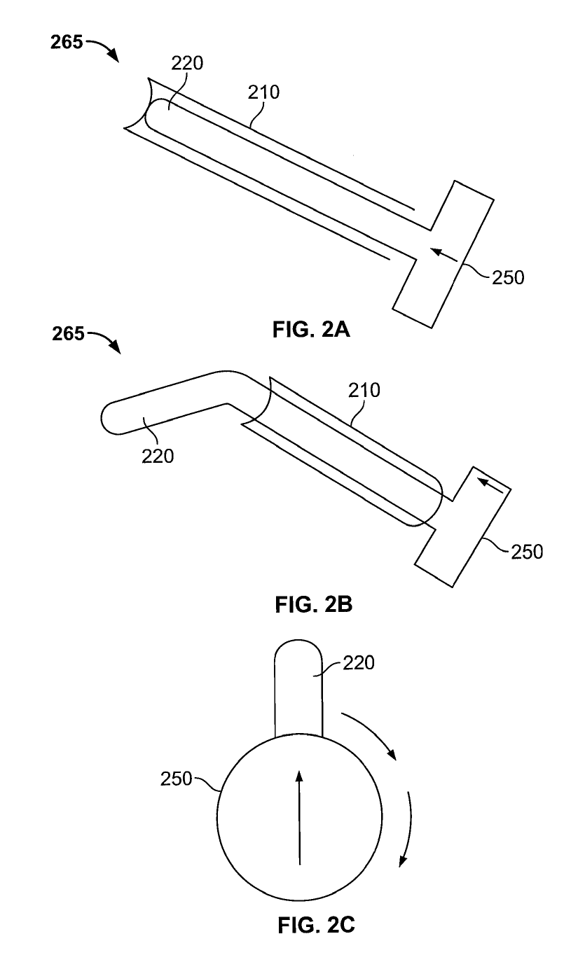

Referring to FIGS. 2A-2C, a heat delivery device 265 includes a steering mechanism in the form of a pre-bent guide wire 220 over which a heating member (not shown) of the heat delivery device can be advanced. The steering mechanism includes a knob or dial 250 for actively steering the guide wire by rotating the guide wire about its longitudinal axis. In an undeployed state (FIG. 2A), guide wire 220 is located entirely within a sheath 210 of the heat delivery device 265.

Guide wire 220, formed, for example, from nitinol, has a predetermined bent shape. In the undeployed state, sheath 210 constrains the guide wire to the shape of the sheath. Upon deployment from the sheath, as shown in FIG. 2B, guide wire 220 assumes its predetermined bent shape.

Guide wire 220 is navigated along the Y-Z plane by rotating dial 250, which in turn rotates the guide wire, as shown in FIG. 2C. Rotation of guide wire 220 before deployment of the guide wire controls the direction in which the guide wire travels when deployed from sheath 210. Dial 250 can alternatively or additionally be used to rotate guide wire 220 after deployment of the guide wire from sheath 210. Once guide wire 220 is positioned, a heating member is advanced along the guide wire to the treatment site.

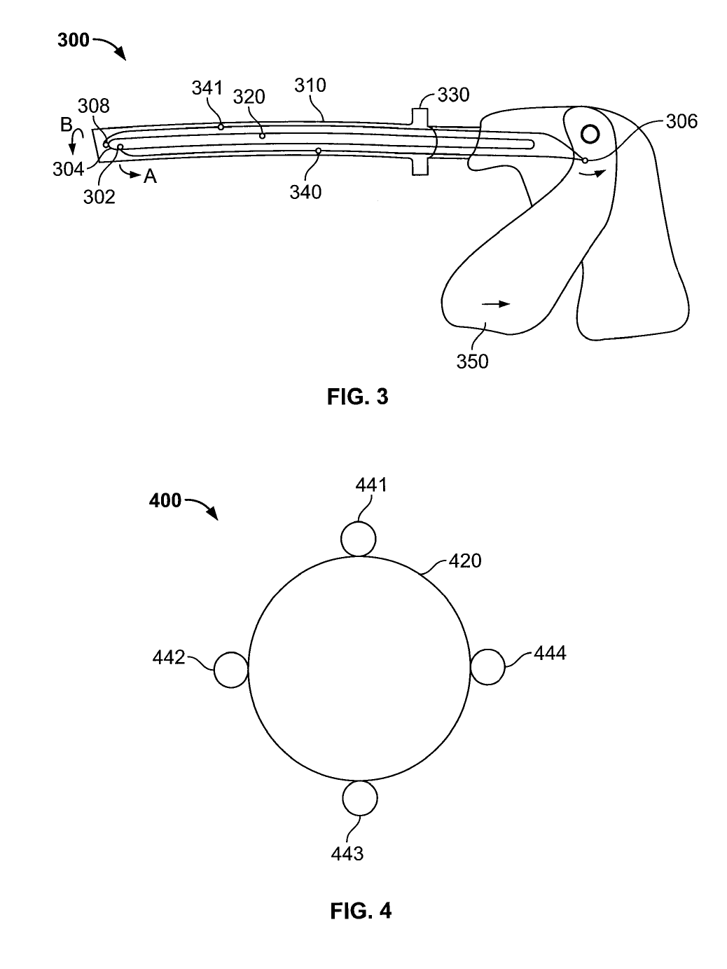

Rather than employing a guide wire having a predetermined bent shape, the heat delivery device 300 of FIG. 3 has an active steering mechanism that uses pull wires 340 and 341. The steering mechanism includes a guide catheter 320 positioned within a sheath 310. Pull wire 340 is attached at its end 302 to a first side of a distal portion 304 of guide catheter 320. Pull wire 341 is attached at its end 302 to a second side of the distal portion 304 of guide catheter 320, where the second side is at a location 90° from the first side. Both pull wires 340 and 341 are attached at their opposite end 306 to a handle 350. Activation of the handle 350 results in the distal portion of guide catheter 320 bending in direction A (i.e., down), if pull wire 340 is contracted, and in direction B (i.e., to one side), if pull wire 341 is contracted. When neither pull wire is contracted, i.e., when the handle is not activated), the guide catheter 320 travels in a straight direction. To cause the guide catheter 320 to travel in a direction opposite of directions A or B (i.e., travel up or to the other side), the heat-delivery device can be rotated 180°. Additionally, to cause the guide catheter 320 to travel in a horizontal direction, such as at 45° or 60° from the original orientation, the heat-delivery device can be similarly rotated to achieve this result.

As shown in FIG. 3, a directional arrow can be indicated on the proximal end (operator's hand end) of an active steering mechanism, such as handle 350, to indicate the direction in which the device will travel when the active steering component is activated. Additionally or alternatively, a directional arrow can similarly be indicated directly on the proximal end of a device or guide catheter.

Sheath 310 is made, for example, of plastic, and is attached to handle 350 with, for example, a standard luer connection 330. The sheath 310 and guide catheter 320 (with the pull wires 340 and 341 attached) is inserted into an intervertebral disc using, for example, a 17 gauge introducer needle (not shown). As the sheath 310 and guide catheter 320 are advanced into the disc, active steering is achieved by pulling the handle 350 to contract the pull wires 340 and 341 and bend the tip of the guide catheter 320, thus causing deflection in the A and/or B directions. In the relaxed state (i.e., when the handle is not engaged), the guide catheter 320 and sheath 310 are advanced in a straight direction. Once the catheter has been successfully placed at a treatment site, the luer connection 330 can be released and the sheath 310 is removed. The guide catheter 320 remains to provide a channel for placement of treatment devices, such as a heating member of the heat-delivery device.

In FIG. 4, a guide catheter 420 of an active steering mechanism of heat delivery device 400 includes four pull wires 441-444 located in the four quadrants of the guide catheter 420 running co-axial with the guide catheter 420. The wires are engaged and disengaged to contract and stretch in pairs such as, for example, wire pair 441 and 443 and wire pair 442 and 444. The four pull wires provide a full range of motion in two dimensions. The two wire pairs can be activated sequentially or simultaneously.

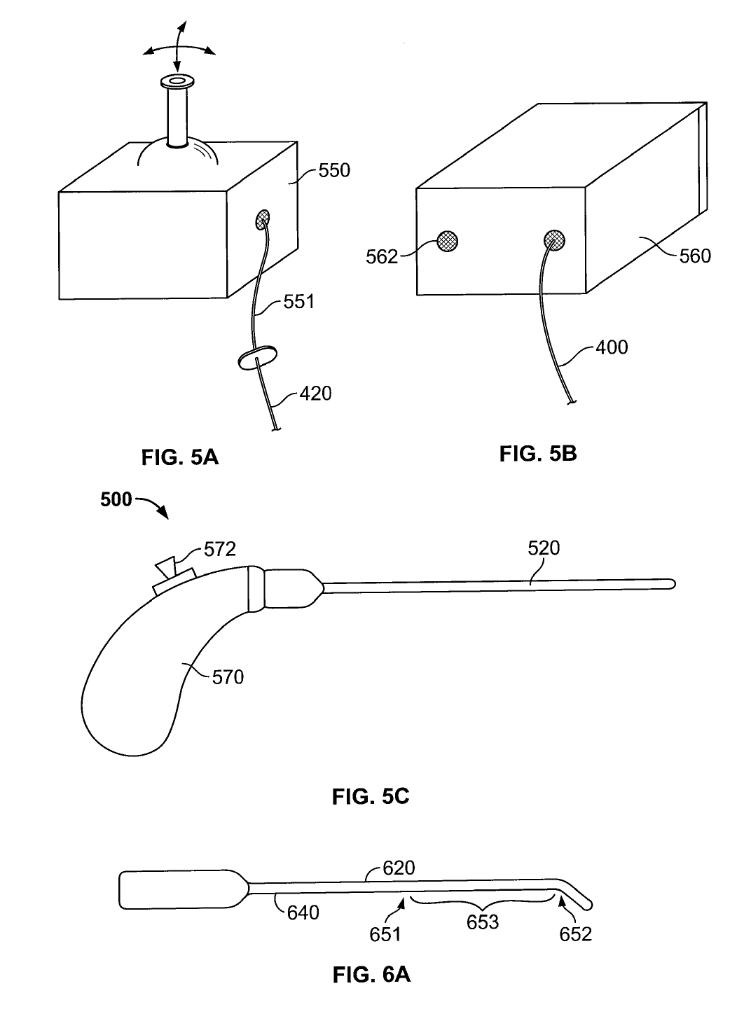

Referring to FIG. 5A, the pull wires can be controlled by a joystick 550 coupled to the guide catheter 420 by a cable 551. Joystick 550 can be moved left, right, up, down and diagonally to actively steer the guide catheter 420. Alternatively, the pull wires can be controlled by a joystick or buttons 562 on a generator 560 (FIG. 5B) to which the heat delivery device 400 is connected. A joystick 572 can be integrated into the handle 570 of a heat delivery device 500, as shown in FIG. 5C, to actively steer a guide catheter 520.

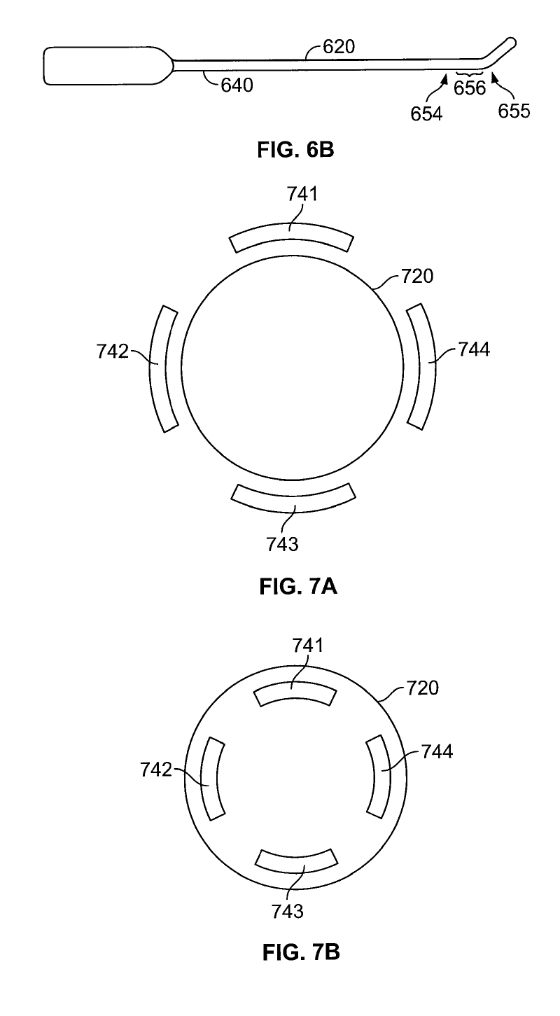

Referring to FIGS. 6A and 6B, the sensitivity of pull wire activation can be controlled by adjusting the length and placement of a pull wire 640 connected to a guide catheter 620. For example, FIG. 6A shows wire 640 connected to guide catheter 620 along a length 653 of the catheter between locations 651 and 652. By connecting the wire to the guide catheter over a large portion of the guide catheter, a large section of the guide catheter 620 deflects when the wire 640 is activated (e.g., contracted). As such, the guide catheter 620 is sensitive to slight contractions of the wire 640, and thus turns abruptly without a large amount of steering by the operator.

In another example, FIG. 6B shows wire 640 connected to guide catheter 620 along a shorter length 656 of the catheter between locations 654 and 655. By connecting the wire to the guide catheter over a smaller portion of the guide catheter, a smaller section of the guide catheter 620 deflects when the wire 640 is contracted. As such, and in contrast to the configuration shown in FIG. 6A, the navigation of guide catheter 620 is not as sensitive to small amounts of steering.

In FIGS. 7A and 7B, navigation of a guide catheter 720 is controlled by thin strips 741-744 rather than wires that contract and stretch to bend the catheter. Strips 741-744 can be located outside (FIG. 7A) or within (FIG. 7B) guide catheter 720. Strips 741-744 can be controlled to actively steering the guide catheter 720 using, for example, a handle, dial, or joystick, as described above.

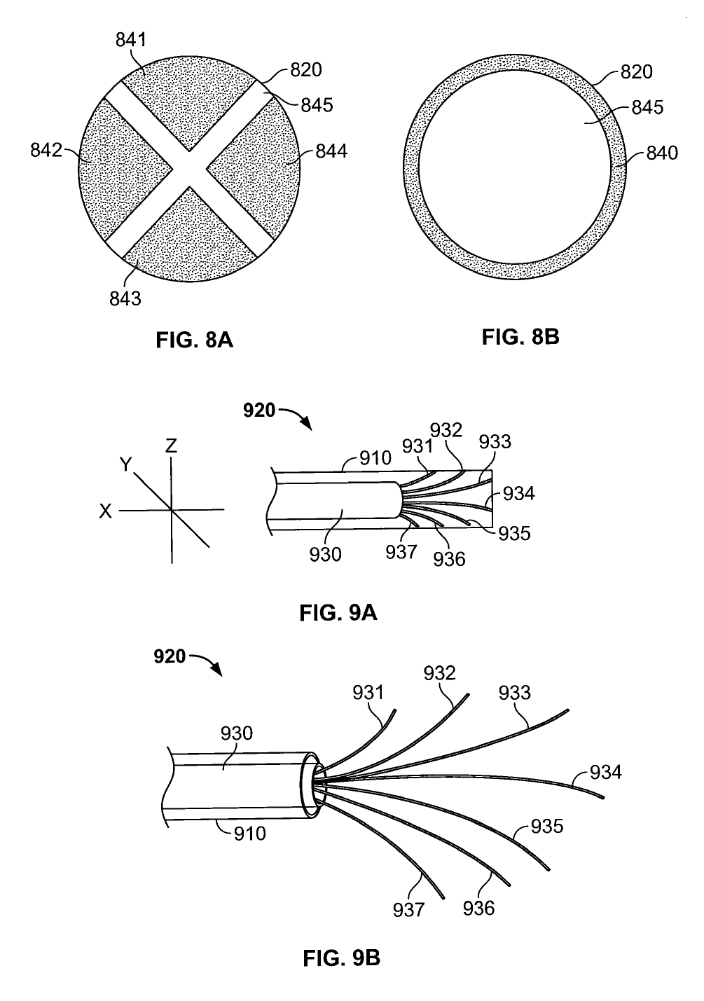

Referring to FIGS. 8A and 8B, rather than wires or strips, a guide catheter 820 can be controlled by providing electricity to a conductive fluid, such as a liquid or gel, contained within the walls of the guide catheter 820. In FIG. 8A, the tube that defines guide catheter 820 has four quadrants 841-844, each filled with a conductive fluid and separated by portion 845 that, in some implementations, is filled with an insulating material. In FIG. 8B, the tube that defines guide catheter 820 includes an open core 845 surrounded by a ring of conductive fluid 840. In an alternative implementation, a tube is completely filled with a conductive fluid. When electricity is applied to the fluid (e.g., along the outside or inside walls of the guide catheter 820 or directly into the fluid), the guide catheter bends in a particular direction. In this manner, the operator can apply an amount of electricity to actively control the amount and direction in which the guide catheter will bend, and thus actively steer the guide catheter to a desired location within an intervertebral disc.

Actively navigating a guide catheter, or sheath into an intervertebral disc allows for placement of different devices into the intervertebral disc in order for different functions to be performed. For example, a sharp device can be placed into the sheath in order to penetrate tough tissue such as occurs within the nucleus of a degenerated disc or to penetrate the annulus at the site of a fissure to gain access to the site of pathology for treatment. The sharp device can then be removed from the sheath and another device, such as, for example, a heating member, can be inserted to apply heat to a treatment site in order to cause denervation of a fissure. In another example, a fiber optic device can be placed within the sheath to visualize the disc for purposes of identifying the site of a fissure or inspecting the state of degeneration of the disc. Additionally, an auger or other resecting type of device, including an aspirator, can be placed within the sheath and used to remove tissue. Material, such as an enzyme to digest tissue, a sealant to repair the fissure, or a pharmaceutical agent(s) to treat the disc can also be introduced into the disc through the sheath.

In addition to precise placement via active steerability, the ability to preferentially penetrate tissues could be necessary as the disc often has areas of relatively dense tissue that can be difficult to penetrate. Coming into contact with this dense tissue can result in deflection from the desired trajectory. Thus, an active steering mechanism can also provide functionality for penetrating dense tissue to ensure that precise placement is achieved.

A sheath or guide catheter desirably fits through a 17 gauge, or smaller, introducer needle. Using a 17 gauge needle is beneficial because large holes placed in the disc have been demonstrated to lead to degeneration. Thus, sizing of components and devices placed into an intervertebral disc through a sheath are likely to be constrained. However, providing for interchangeability of components having different functions reduces the size constraints on those components. In the configurations described above, one component is placed within the sheath at a given time. As such, each component to be used can be larger than if the component were merely a sub-component of a multifunctional device that was inserted into the body once to perform more than one function. Alternatively, the components are sub-components of a multifunctional device.

In addition to treating discogenic back pain, a benefit for intradiscal procedures intended to treat radicular symptoms due to herniated discs can also be achieved because the placement of the device provides targeted therapy.

In addition to providing targeted placement of a device, such as a heat-delivery device, within an intervertebral disc, a large heating profile can be provided to apply heat to a substantial portion of the intervertebral disc or a treatment site within the three dimensional disc. To do so, efficacious configurations of a heating element of a heat-delivery device are used to provide a large heating profile over a large volume. In one example, the heating element includes a series of wires that deploy from a catheter and spread out. The individual wires can then emit monopolar or bipolar radiofrequency (RF) energy, resistive heat or other energy to induce heating. Such deployment can be accomplished by, for example, using shape memory metals that assume a predetermined configuration once extended beyond the confines of a sheath. Spring-loaded, or spring-biased, configurations can also be used. In another example, the heating element includes a broad, flat surface that spreads out once extended beyond the confines of a sheath.

Enlarging the heating profile to deliver heat to a broader volume within a disc is useful because of the three dimensional structure of the disc. An intervertebral disc has a volume that extends along the X, Y and Z axes. The disc typically ranges in height (along the Z-axis) from about 0.5 to 1 cm. By enlarging the area of heat delivery, the ability to ensure heating at the site of pathology is improved by, for example, being able to fully treat a pathology even if positioning of a heat-delivery device directly at the treatment site is not achieved. More particularly, by providing a large heating profile over a large volume of a disc, all, or a substantial portion of, a fissure, including along the z-axis of the disc receives heat adequate to cause denervation.

Referring to FIGS. 9A and 9B, a heat-delivery device 920 includes an actively steerable sheath 910 and a heating member 930 located within the sheath 910. Heating member 930 includes a series of heating elements 931-937 in the form of shaped wires or electrodes for providing monopolar or bipolar energy to treat a fissure by heating the fissure.

Heating member 930 can be advanced and retracted (along axis X) within sheath 910 between an undeployed stated (FIG. 9A) and a deployed state (FIG. 9B) with heating elements 931-937 extending from sheath 910 in the deployed state. In the deployed state, the wires of heating member 930 are exposed and fan out along the Y and Z axes, such that wires extending from the middle of heating element 930 include a straight, or nearly straight, shape, while wires on either side of the middle wires are bent away from the center of heat-delivery device 920. Such spreading out, and bending, of the wires upon deployment is accomplished, for example, using shape memory metals that assume a predetermined configuration once extended beyond the confines of the sheath. Spring-loaded, or spring-biased, configurations also can be used. The distal tips of the wires (in an implementation where a portion of each wire is insulated) or the entire wire (in an implementation where the entirety of the wires is exposed or uninsulated) extend beyond sheath 910 in the deployed state. Sheath 910 can be actively steerable as discussed above. Alternatively, or in addition, heating member 930 is actively steerable. Sheath 910 is, for example, a tube of braid reinforced polyimide having an inner diameter of about 0.028 inches and an outer diameter of about 0.036 inches.

By providing heating elements that include wires that fan out in the deployed state, the heat-delivery device 920 provides a large heating profile to cover a large volume of an intervertebral disc. The large heating profile covers a volume of the intervertebral disc that includes three dimensions, such that heat may be applied along the X, Y and Z axes of a disc or a treatment site within a disc.

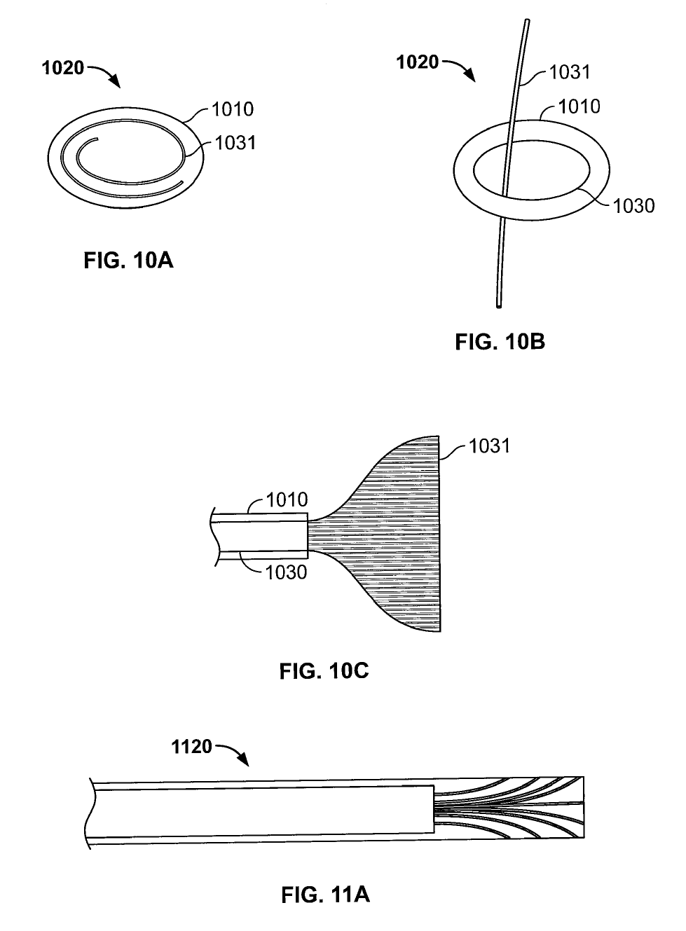

Referring to FIGS. 10A-10C, a heat delivery device 1020 includes a sheath 1010 and a heating member 1030. Heating member 1030 includes a heating element 1031 that has an enlarged flat profile in a deployed state (FIGS. 10B and 10C) to cover a large volume of the intervertebral disc. In the undeployed state (FIG. 10A), heating element 1031 is coiled completely within sheath 1010. In other implementations, heating element 1031 can adopt other shapes in the deployed and undeployed states. The sheath and/or the heating member can be actively steerable.

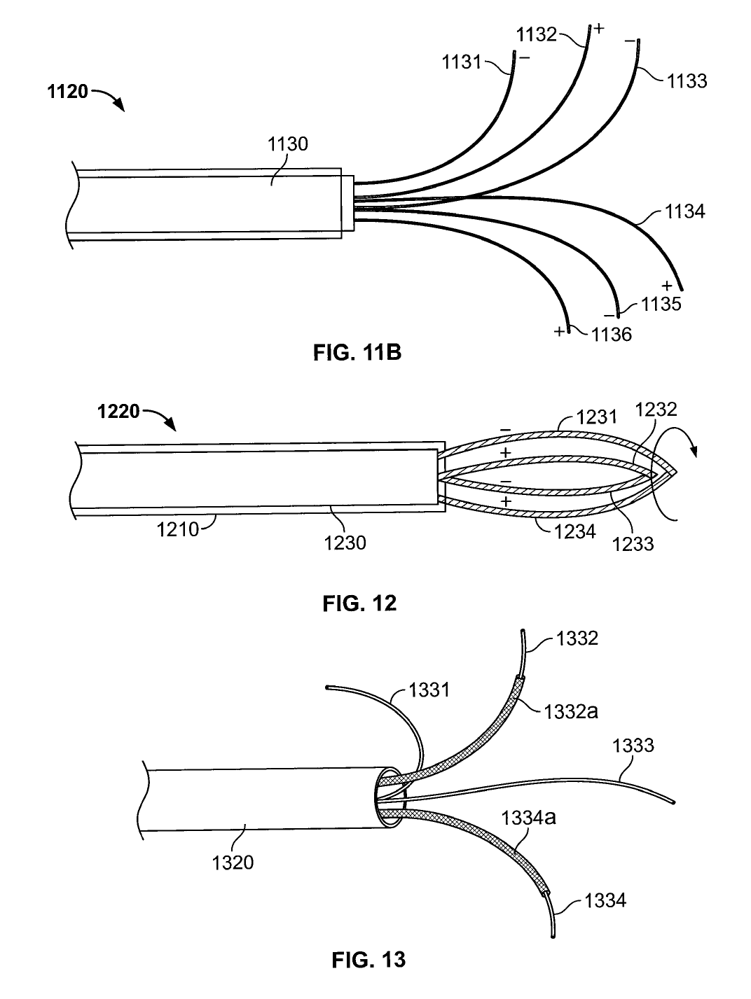

Referring to FIGS. 11A and 11B, a heat-delivery device 1120, such as shown in FIGS. 9A and 9B includes heating element electrodes 1131-1136 alternatively charged with positive and negative voltage in a bipolar configuration. For example, electrodes 1131, 1133 and 1135 are negatively charged, while electrodes 1132, 1134 and 1136 are positively charged. In other implementations, the electrodes are configured in a monopolar fashion, such that the electrodes are all positively charged and a ground pad is placed on the patient's skin.

Referring to FIG. 12, a heating member 1230 of a heat-delivery device 1220 includes a series of heating wires 1231-1231 that extend out from a sheath 1210. Wires 1231-1234 form a basket configuration. In some implementations, the basket rotates in a clockwise direction, a counter-clockwise direction, or both. In some implementations, the rotation can be initiated by engaging an on/off switch, which causes power to be applied to a rotating mechanism, and in turn causes the basket to rotate in a selected direction. Alternatively, a manual crank or dial is turned in a selected direction to cause the basket to rotate in the selected direction. Rotating the basket enlarges the heating profile provided by heating member 1230.

The wires are alternatively charged with positive and negative voltage in a bipolar configuration that produces a concentrated heating field within and around the basket. Alternatively, a monopolar configuration is used, such that all of wires 1231-1234 are positively charged and heat flows from the wires to a location of a ground pad placed on the patient's skin.

In FIG. 13, bipolar electrodes 1331-1334 of a heat-delivery device 1320 are partially insulated (electrodes 1332 and 1334) with, for example, thin polyester shrink tubing 1332a and 1334a, forming positive electrodes to provide high voltage, or bare, i.e., uninsulated, (electrodes 1331 and 1333) to provide ground-return.

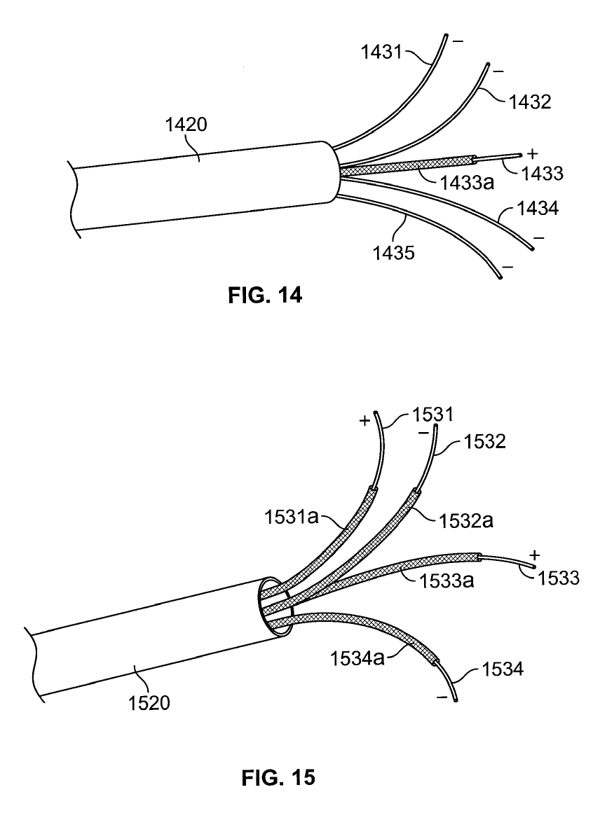

In FIG. 14, a heat-delivery device 1420 includes five wire electrodes 1431-1435. The center prong 1433 has a straight shape and is insulated with insulation 1433a up to, for example, 0.2 inches from the distal tip. The four side prongs 1431, 1432, 1434 and 1435 are uninsulated. Heating member 1430 is configured in a bipolar manner, such that prong 1433 provides high voltage, while prongs 1431, 1432, 1434 and 1435 are used as ground returns. In a bipolar configuration, the small surface area of the uninsulated portion of center prong 1433 limits the size of the lesion because the current density is relatively high at the uninsulated portion.

Referring to FIG. 15, a heat-delivery device 1520 includes bipolar electrode wires 1531-1534 that are all at least partially insulated. Wires 1531-1534 are each insulated, shown at 1531a-1534a, up to an exposed portion at the distal tip. The exposed portion at the distal tip of the prongs is, for example, 0.2 inches in length. Such insulation provides surface area at the tip of each of the multiple positively and negatively (or grounded) charged electrodes. This configuration causes a lesion area having a larger diameter to be produced because of the high current density at the tip of all four prongs 1531-1534. By increasing the size of the uninsulated tip, the size of the lesions produced is controlled. However, more electrical surface area requires more power to achieve adequate heating to cause the lesion.

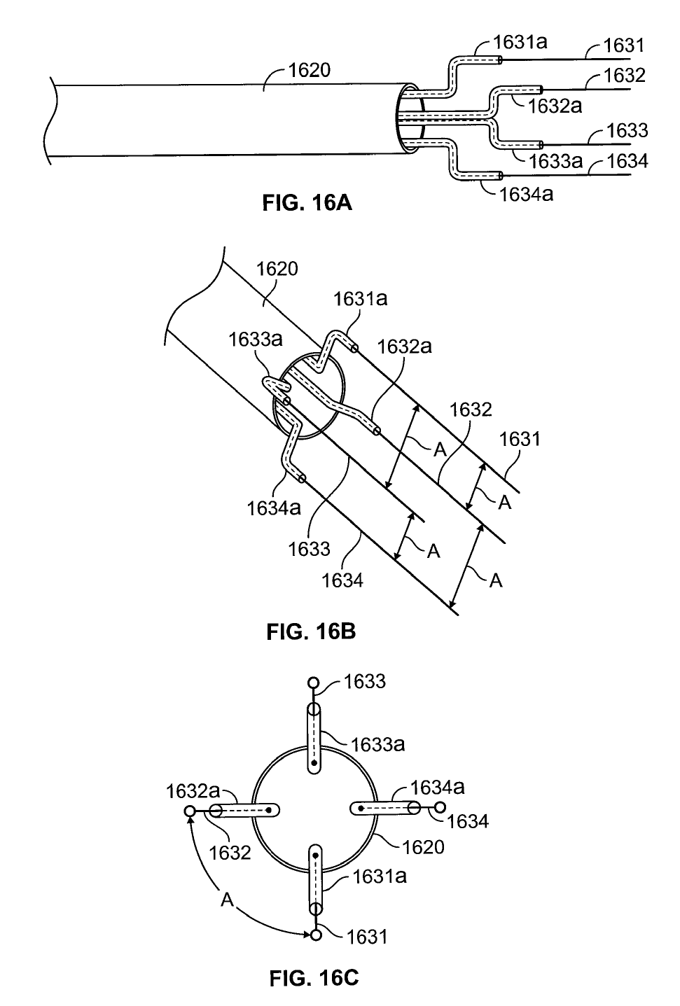

In FIGS. 16A-16C, a heat-delivery device 1620 includes wire electrode prongs 1631-1634 having insulated portions 1631a-1634a. Prongs 1631-1634 have a straight, rather than fanned-out shape, and are configured to be parallel to one another. As such, rather than deploying in a manner where the distance between the prongs varies along the length of the prongs, the distance A between each of prongs 1631-1634 remains constant along the uninsulated portions in the deployed state. In this manner, electrical current flowing between (in a bipolar configuration), or from (in a monopolar configuration), the prongs is more uniformly distributed because the electrical path remains more constant. The insulated portions 1631a-1634a cover the non-parallel portion of each of prongs 1631-1634. As described above, the configuration of the prongs or electrodes that form the heating member affects the heating profile provided and thus the efficacy of treatment (e.g., location and concentration of lesions). For example, by adjusting the size of the uninsulated portion of the prongs (i.e., the amount of insulation on each prong), the lesion size and location can be controlled. The amount of energy delivered also affects and controls the production of lesions in that the more energy applied, the more lesions are produced. There is also a time/temperature effect such that longer heating times will generally enhance the tissue effect, producing more lesions. Varying the diameter of the prongs also affects the lesion shape and distribution.

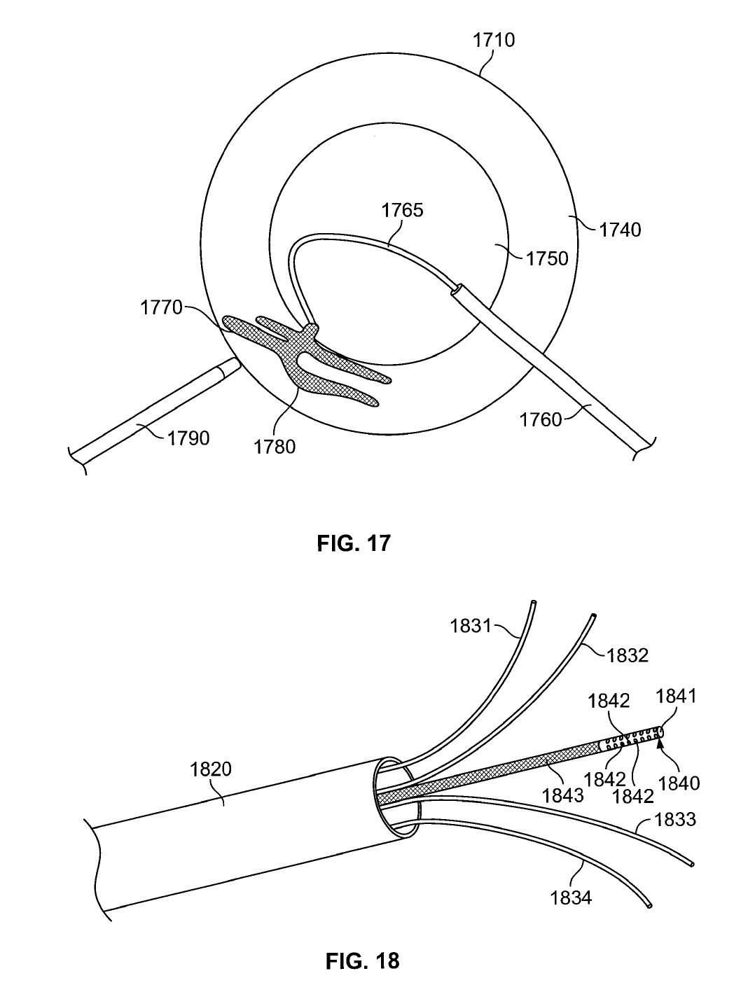

Referring to FIGS. 17 and 18, a fluid, such as an electrically conductive material, can be injected into a treatment site to aid in heat transfer and denervation. In some implementations, a larger heating profile is provided by using conductive materials, in addition to, or instead of, employing the various heating element configurations described above. If the tissue to be treated has a high impedance, then a smaller amount of heat delivery (resulting in a smaller lesion size) is required than if the tissue has a low impedance. In one embodiment, the impedance of the tissue at a treatment site can be temporarily decreased by injecting a conductive material, such as, for example, saline. By temporarily decreasing impedance, the conductive material can temporarily increase conductivity at the treatment site. Heat or energy is then applied to the tissue to which the conductive material has been introduced in order to produce a relatively larger lesion than would have resulted without the injection of the conductive material.

In FIG. 17, an introducer needle 1760 has been inserted into the intervertebral disc and a device, such as, for example, a heat-delivery and conductive material provisioning device 1765, has been navigated to a treatment site. The treatment site is the site of fissure 1770. A conductive material 1780 is introduced into the fissure 1770 via device 1765.

The conductive material 1780 can be, for example, saline. Preferably, the saline is provided at a desired location within a disc (i.e., treatment site) and not at other, unrelated, locations. To limit the introduction of saline to the desired location, alternative or additional materials can be used, such as, for example, a thickened electrolytic media. The thickened electrolytic media can ‘set-up’ at the treatment site to form a cast within a fissure. In other implementations, the conductive material 1780 is a radiopaque dye. Radiopaque dye can be used to provide fluoroscopic visualization or to aid visual localization through a fiber optic placed into the intervertebral disc subsequent to introduction of the dye. Once the conductive material 1780 has been introduced into the fissure 1770, a heat-delivery device can be used to apply heat to the conductive material, and thus heat the fissure.

In one embodiment, a fissure is analyzed using discography to determine if it is leaky. This determination is an important first step in determining whether to introduce a conductive material. If the fissure is leaky, a conductive material introduced into the fissure can escape the disc and not provide the desired level of decrease in impedance and increase in conductivity.

To appropriately heat a treatment site, temperatures greater than 45° C. over a large volume are applied to substantially all of the treatment site. Additionally, or alternatively, temperatures greater than 45° C. are attained over a smaller volume. Control of the temperature of heat provided by the heat-delivery device is based on measurements of temperature using a thermocouple (TC) or similar sensor. Based on a TC reading, generator power can be modulated to provide an appropriate amount of heat.

Referring to FIG. 17, a thermocouple (TC) 1790 is placed outside the annulus 1750 to measure the temperature of heat applied to a conductive material 1780 within fissure 1770. TC 1790 is used to monitor, and thus determine control and alteration of, the temperature and amount of heat applied to the conductive material 1780. In one implementation, and as shown in FIG. 17, a TC is placed directly at the site of pathology (e.g., on the outer annulus at the site of a fissure) using, for example, an introducer needle positioned under fluoroscopic guidance. The fissure is localized, using, for example, magnetic resonance imaging (MRI) or discogram techniques. A heat-delivery device is placed intradiscally, actively steered to the site of the fissure on the inner annulus, and used to apply heat until sufficient temperature readings on the external TC are attained. In this approach, the temperature control can be site and/or patient specific. As such, adequate temperature to achieve denervation is provided, yet the technique is safer in that it limits damage to structures that are not intended to receive heat therapy.

In another implementation, the heat-delivery device includes an integral TC mounted to the heat-delivery device. The TC can be positioned on the device to measure the temperature at the site being treated, rather than the temperature at the heating member of the heat-delivery device. This is helpful when the heating member of the heat-delivery device has been placed at a selected distance from the treatment site. Thus, the TC can be positioned on the device at a selected distance from the heating member of the heat-delivery device, where the selected distance is the same distance from the heating member as the distance from the heating member to the treatment site. For example, the TC is placed at a distance proximal or distal along the length of the heat-delivery device such that temperature at a distance from the heating member (i.e., at the same distance as the distance from the heating member to the treatment site) can be measured. This approach helps ensure that adequate heat is provided at the desired location. In yet another implementation, a TC that is not integral to the heat-delivery device can be placed at the treatment site, or at a selected distance from the treatment site, by navigation separate from the steering of the heat-delivery device.

Referring to FIG. 18, a center post 1840 of a device 1820 including wire electrodes 1831-1834, can be used to inject a conductive material, such as, for example, saline, into an intervertebral disc. The center post 1840 includes an inner bore 1841 and is insulated, shown at 1843, except for the distal tip. The distal tip is perforated, shown at 1842, and configured to allow diffusion of the conductive material, provided through the inner bore, throughout tissue at a treatment site (e.g., a location where a lesion is desired). The center post 1840 also can be energized, with the other prongs 1831-1834 serving as ground returns.

A device for applying heat and/or a conductive material to a treatment site within an intervertebral disc is sturdy, stiff and has a high flexural modulus. These attributes enable the device to be navigated through the dense tissue that is typical of the annular wall of, and tissue within, an intervertebral disc. Furthermore, the device can be configured in size to be commensurate with the areas in the intervertebral disc through which the device will be navigated and the volume of the intervertebral disc.

In some implementations, the heat-delivery devices are configured to apply RF energy, having a frequency of, for example, 460 kHz. Other implementations are configured to provide a higher frequency, such as, for example, 1 MHz.

Although this disclosure is primarily focused on the treatment of discogenic pain, radicular pain also can be treated using the techniques disclosed. For example, herniated disc tissue can be removed and shrunk to reduce the resultant insult (e.g., compression and sensitization) on the nerve root and effectively relieve radicular symptoms.

A number of embodiments have been described. Nevertheless, it will be understood that various modifications may be made. For example, elements of different implementations may be combined, supplemented, or removed to produce other implementations. In particular, features from an implementation that provides a large heating profile can be combined with features from an implementation that provides targeted placement of a device using an active steering mechanism. Accordingly, these and other features are within the scope of the following claims.

What is claimed is:

1. A method for treating an intervertebral disc comprising:
actively steering a heat-delivery device to a region of a treatment site within the intervertebral disc, wherein the treatment site is three dimensional and the steering navigates the heat-delivery device in at least two dimensions; and
applying heat to the region of the treatment site using the heat-delivery device.

2. The method of claim 1 wherein actively steering the heat-delivery device includes turning a knob positioned external to a patient to navigate the heat-delivery device in two dimensions.

3. The method of claim 1 wherein actively steering the heat-delivery device includes activating a handle positioned external to a patient to navigate the heat-delivery device in two dimensions.

4. The method of claim 1 wherein the heat-delivery device comprises a guide wire and a heating element, and actively steering the heat-delivery device comprises:
actively steering the guide wire to the region of the treatment site within the intervertebral disc; and
advancing the heating element to the region of the treatment site over the guide wire.

5. The method of claim 1 wherein the heat-delivery device comprises a sheath and a heating element, and actively steering the heat-delivery device comprises:
actively steering the sheath to the region of the treatment site within the intervertebral disc; and
advancing the heating element to the region of the treatment site through the sheath.

6. The method of claim 5 further comprising:
removing the heating element from the sheath; and
advancing a second device, having a function other than heat-delivery, to the treatment site through the sheath.

7. The method of claim 1 wherein applying heat to the region of the treatment site comprises applying heat to substantially all of the treatment site.

8. The method of claim 7 wherein applying heat to the region of the treatment site comprises applying heat to the treatment site using a heating element of the heat-delivery device having a three dimensional shape that corresponds to a three dimensional shape of the treatment site.

9. The method of claim 7 wherein applying heat to the region of the treatment site comprises applying heat at a location that is at a distance from the treatment site.

10. The method of claim 1 further comprising:
injecting a conductive material into the treatment site; and
applying heat to the conductive material in the treatment site.

11. The method of claim 10 wherein the conductive material comprises saline.

12. The method of claim 1 further comprising monitoring the temperature of the heat applied to the treatment site.

13. The method of claim 12 further comprising placing a thermocouple at the outer wall of the annulus of the intervertebral disc to monitor the temperature of the heat applied to the treatment site, wherein applying heat to the treatment site comprises applying heat to the inner wall of the annulus of the intervertebral disc.

14. The method of claim 12 wherein actively steering a heat-delivery device to the region of the treatment site comprises actively steering the heat-delivery device to a location at a distance from the treatment site, and further comprising placing a thermocouple at a distance from the heat-delivery device that mimics the distance from the heat-delivery device to the treatment site to monitor the temperature of the heat applied to the treatment site.

15. The method of claim 12 wherein monitoring the temperature comprises navigating a thermocouple to a location for monitoring the temperature of the heat applied separately from the navigating of the heat-delivery device.

16. The method of claim 1 wherein applying heat to the treatment site comprises applying heat having a temperature of greater than 45° C. to the treatment site.

17. The method of claim 1 wherein applying heat to the treatment site comprises applying heat to the treatment site with a heating element of the heat-delivery device.

18. The method of claim 17 wherein applying heat to the treatment site comprises applying heat to the treatment site in a bipolar configuration.

19. The method of claim 18 wherein heat is applied in a bipolar configuration by at least two electrodes that are alternatively charged with negative and positive voltage.

20. The method of claim 17 further comprising placing a ground pad on a patient's skin, and wherein applying heat to the treatment site comprises applying heat to the treatment site in a monopolar configuration.

21. The method of claim 20 wherein heat is applied in a monopolar configuration by at least two electrodes that are charged with positive voltage.

22. The method of claim 17 wherein applying heat to the treatment site with the heating element comprises applying heat to the treatment site with electrodes formed into a basket configuration.

23. The method of claim 17 wherein applying heat to the treatment site with the heating element comprises applying heat to the treatment site with an electrode that is coiled inside a sheath in an undeployed state and is extended into the intervertebral disc to form a flat shape in a deployed shape.

24. The method of claim 17 wherein applying heat to the treatment site with the heating element comprises applying heat to the treatment site with at least two electrodes, wherein a distance between the electrodes is constant along the length of the electrodes.

25. The method of claim 17 further comprising injecting a fluid into the treatment site, wherein the heating element is configured to inject the fluid into the treatment site.

26. An intervertebral disc treatment device comprising:
a heat-delivery device configured to encompass a three dimensional volume of the intervertebral disc to provide heat to a three dimensional treatment site within the intervertebral disc; and
an active steering mechanism configured to enable navigation of the heat-delivery device in at least two dimensions.

27. The device of claim 26 wherein the active steering mechanism comprises a pre-bent guide wire.

28. The device of claim 26 wherein the active steering mechanism comprises:
a guide catheter; and
pull elements attached to the guide catheter for bending the guide catheter.

29. The device of claim 26 wherein the active steering mechanism comprises:
a guide catheter; and
a conductive fluid contained within the guide catheter to which electricity can be applied to bend the guide catheter.

30. The device of claim 26 wherein the heat-delivery device comprises a heating member that includes electrodes that expand when deployed within the intervertebral disc to encompass the three dimensional volume of the disc.

31. The device of claim 30 wherein the heating member comprises electrodes configured to fan out in the deployed state.

32. The device of claim 30 wherein the heating member comprises electrodes configured in the shape of a basket.

33. The device of claim 26 wherein the heat-delivery device comprises:
a sheath; and
a heating member including an electrode coiled within the sheath in an undeployed state.
